(19) United States
(12) Patent Application Publication
(10) Pub. No.: US 2005/0244465 A1
(43) Pub. Date: Nov. 3, 2005

(54) Drug delivery systems and methods for treatment of an eye

(75) Inventors: Thierry Nivaggioli, Los Altos Hills, CA (US); Victoria Rogers, San Bruno, CA (US)

Correspondence Address: STOUT, UXA, BUYAN & MULLINS LLP, 4 VENTURE, SUITE 300, IRVINE, CA, 92618, US

(73) Assignee: Allergan, Inc., Irvine, CA (US)

(21) Appl. No.: 10/837,291

(22) Filed: Apr. 30, 2004

(51) Int. Cl. A61K 9/00

(52) U.S. Cl. 424/427

(57) ABSTRACT

Systems and methods are provided for treatment of an eye. The systems generally include controlled, delayed and or sustained release bioerodible implantable elements having multiple layers of different materials and/or different concentrations of materials. The elements generally include an inner layer, or core, including a therapeutic agent, and one or more outer layers made of polymeric materials, for example substantially pure polymeric materials. The core may include a polymeric material combined with an active agent beneficial in treating a condition of an eye. The elements may be structured such that surfaces of the core are exposed to the physiological environment of the eye when initially implanted within the eye, or may be structured such that the core is entirely enclosed within the outer layers when initially implanted within the eye.

### BACKGROUND

The present invention generally relates to drug delivery systems for controlled, sustained and/or delayed drug release in an eye, and more specifically relates to multilayered implants, and methods of using such implants, for treatment of an eye, for example, a mammalian eye.

Solid pharmaceutically active implants that provide controlled release, for example, sustained release, of an active ingredient are able to provide a relatively uniform concentration of active ingredients in the body. Implants are particularly useful for providing a high local concentration at a particular target site for extended periods of time. Additionally, sustained release forms may reduce the number of doses of the drug required to be effective in treatment of a condition, and often reduce the occurrence of side effects and/or inconsistency in drug concentration found with traditional drug therapies.

However, many current formulations of sustained release implants have been found to have release profiles that do not provide relatively constant or consistent level of active component. For example, certain controlled release implants that are designed to provide consistent, sustained release, actually show little release until nearly complete erosion of the implant, at which time there can be a dumping of the drug. These implants are known to exhibit undesirable sigmoidal, or S-shaped, release profiles, wherein there is a clear inconsistency in the release rate of the drug over time. Other implants, especially those including highly soluble drugs, exhibit an initial burst of the drug wherein a majority of the drug is released within a very short time of being implanted.

It would be advantageous to provide eye implantable drug delivery systems, and methods of using such systems, having more consistent sustained release rates, delayed release rates, or other controlled and/or modified release rates for effective treatment of ocular diseases and disorders.

Macular degeneration, such as age related macular degeneration (“AMD”) is the leading cause of blindness in the world. It is estimated that thirteen million Americans have evidence of macular degeneration. Macular degeneration results in a break down the macula, the light-sensitive part of the retina responsible for the sharp, direct vision needed to read or drive. Central vision is especially affected. Macular degeneration is diagnosed as either dry (atrophic) or wet (exudative). The dry form of macular degeneration is more common than the wet form of macular degeneration, with about 90% of AMD patients being diagnosed with dry AMD. The wet form of the disease usually leads to more serious vision loss. Macular degeneration can produce a slow or sudden painless loss of vision. The cause of macular degeneration is not clear. The dry form of AMD may result from the aging and thinning of macular tissues, depositing of pigment in the macula, or a combination of the two processes. With wet AMD, new blood vessels grow beneath the retina and leak blood and fluid. This leakage causes retinal cells to die and creates blind spots in central vision. Current treatments for macular degeneration are generally limited to those aimed at preventing further progression of the disease. For example, laser photocoagulation is used to destroy blood vessels that have encroached on the macula.

Macular edema (“ME”) can result in a swelling of the macula. The edema is caused by fluid leaking from retinal blood vessels. Blood leaks out of the weak vessel walls into a very small area of the macula which is rich in cones, the nerve endings that detect color and from which daytime vision depends. Blurring then occurs in the middle or just to the side of the central visual field. Visual loss can progress over a period of months. Retinal blood vessel obstruction, eye inflammation, and age-related macular degeneration have all been associated with macular edema. The macula may also be affected by swelling following cataract extraction. Current treatment for ME includes topical anti-inflammatory drops. In some cases, medication is injected near the back of the eye for a more concentrated effect. Oral medications are also sometimes prescribed.

Glaucoma is a serious ocular condition characterized by increased ocular pressure and loss of retinal ganglion cells. Damage caused by glaucoma is thought to be irreversible. Current treatments for early stage glaucoma usually involve therapeutic eyedrops and oral medications used to lower ocular pressure.

Diabetic retinopathy is characterized by angiogenesis. Small blood vessels on the retina of the eye are damaged, resulting in the growth of abnormal blood vessels which proliferate and eventually leak and blur or otherwise obscure vision. Laser surgery is the current mainstay of treatment for diabetic retinopathy. Advanced proliferative diabetic retinopathy may be treated by vitrectomy, which includes removal of a portion of the vitreous and replacement with a clear replacement material. In any event, early treatment of diabetic retinopathy is essential to preventing permanent vision loss.

Uveitis involves inflammation of structures of the uvea. Treatment may consist of topical eyedrops or ointments containing cortiosteroids.

Retinitis pigmentosa is characterized by retinal degeneration. Retinitis pigmentosa is considered to be not one disease, but rather a group of diseases with common attributes. Visual problems common to retinitis pigmentosa include tunnel vision field, night blindness, glare problems, double vision and development of cataracts. Currently, there are no standard treatments available for retinitis pigmentosa, though it is believed that increasing intake of Vitamin A may slow progression of the disease.

Topically or orally administered medicinal agents, for example anti-inflammatory (i.e. immunosuppressive) agents, are currently a first line of treatment for many ocular conditions.

A major problem with topical and oral drug administration of drugs in treatment of the eye is the inability of the drug to achieve an adequate (i.e. therapeutic) intraocular concentration.

Systemic glucocorticoid administration is often used alone or in addition to topical glucocorticoids for the treatment of uveitis. However, prolonged exposure to high plasma concentrations (administration of 1 mg/kg/day for 2-3 weeks) of steroid is often necessary so that therapeutic levels can be achieved in the eye.

Unfortunately, these high drug plasma levels commonly lead to systemic side effects such as hypertension, hyperglycemia, increased susceptibility to infection, peptic ulcers, psychosis, and other complications. Cheng C. K. et al. “Intravitreal sustained-release dexamethasone device in the treatment of experimental uveitis”, Invest. Ophthalmol. Vis. Sci. 36:442-53 (1995); Schwartz, B. “The response of ocular pressure to corticosteroids”, Ophthalmol. Clin. North Am. 6:929-89 (1966); Skalka, H. W. et al. “Effect of corticosteroids on cataract formation”, Arch. Ophthalmol. 98:1773-7 (1980); and Renfro, L. et al. “Ocular effects of topical and systemic steroids”, Dermatologic Clinics 10:505-12 (1992).

Additionally, delivery to the eye of a therapeutic amount of an active agent can be difficult, if not impossible, for drugs with short plasma half-lives since the exposure of the drug to intraocular tissues is limited. A more efficient way of delivering a drug to treat an ocular condition is to place the drug directly in the eye.

Techniques such as intravitreal injection of a drug have shown promising results, but due to the short intraocular half-life of active agent, such as glucocorticoids (approximately 3 hours), intravitreal injections must be frequently repeated to maintain a therapeutic drug level. In turn, this repetitive process increases the potential for side effects such as retinal detachment, endophthalmitis, and cataracts. Maurice, D. M. “Micropharmaceutics of the eye”, Ocular Inflammation Ther. 1:97-102 (1983); Olsen, T. W. et al. “Human scleral permeability: effects of age, cryotherapy, transscleral diode laser, and surgical thinning”, Invest. Ophthalmol. Vis. Sci. 36:1893-1903 (1995); and Kwak, H. W. and D'Amico, D. J. “Evaluation of the retinal toxicity and pharmacokinetics of dexamethasone after intravitreal injection”, Arch. Ophthalmol. 110:259-66 (1992).

Additionally, topical, systemic, and periocular glucocorticoid treatment must be monitored closely due to toxicity and the long-term side effects associated with chronic systemic drug exposure sequelae. Rao, N. A. et al. (1997). “Intraocular inflammation and uveitis”, in: Basic and Clinical Science Course (San Francisco: American Academy of Ophthalmology, 1997-1998), Section 9, pp. 57-80, 102-103, 152-156; Schwartz, B. “The response of ocular pressure to corticosteroids”, Ophthalmol. Clin. North Am. 6:929-89 (1966).; Skalka, H. W. and Pichal, J. T. “Effect of corticosteroids on cataract formation” Arch. Ophthalmol. 98:1773-1777 (1980); Renfro, L. and Snow, J. S. “Ocular effects of topical and systemic steroids”, Dermatologic Clinics 10:505-12 (1992).; Bodor, N. et al. “A comparison of intraocular pressure elevating activity of loteprednoletabonate and dexamethasone in rabbits” Current Eye Research 11:525-30 (1992).

What is needed then are more effective systems and methods for treating ocular conditions. The present invention is concerned with and directed to implantable drug delivery systems and methods for treatment of these and other ocular conditions. The present systems and methods are useful for treating an anterior ocular condition, a posterior ocular condition, or an ocular condition which can be characterized as both an anterior ocular condition and a posterior ocular condition.

The following patents and additional publications include disclosure which is relevant to and/or helpful in understanding the present invention.

Weber et al., U.S. patent application Ser. No. 10/246,884, filed on Sep. 18, 2002, having Pub. No. U.S. 2004/0054374 A1, describes apparatus and methods for delivering ocular implants into an eye of a patient.

Wong, U.S. Pat. No. 4,997,652 discloses biodegradable ocular implants, including encapsulated agents, and describes implanting microcapsules comprising hydrocortisone succinate into the posterior segment of the eye.

Wong, U.S. Pat. No. 5,164,188 discloses encapsulated agents for introduction into the suprachoroid of the eye, and describes placing microcapsules and plaques comprising hydrocortisone into the pars plana.

Wong et al., U.S. Pat. Nos. 5,443,505 and 5,766,242 disclose implants comprising active agents for introduction into a suprachoroidal space or an avascular region of the eye, and describes placing microcapsules and plaques comprising hydrocortisone into the pars plana.

Wong et al., U.S. Pat. No. 5,869,079 discloses combinations of hydrophilic and hydrophobic entities in a biodegradable sustained release implant, and describes a polylactic acid polyglycolic acid (PLGA) copolymer implant comprising dexamethasone.

Wong, U.S. Pat. No. 5,824,072 discloses implants for introduction into a suprachoroidal space or an avascular region of the eye, and describes a methylcellulose (i.e. non-biodegradable) implant comprising dexamethasone.

Zhou et al. discloses a multiple-drug implant comprising 5-fluorouridine, triamcinolone, and human recombinant tissue plasminogen activator for intraocular management of proliferative vitreoretinopathy. Zhou, T., et al. “Development of a multiple-drug delivery implant for intraocular management of proliferative vitreoretinopathy”, Journal of Controlled Release 55: pp. 281-295.

Heller, “Biodegradable Polymers in Controlled Drug Delivery”, in: CRC Critical Reviews in Therapeutic Drug Carrier Systems, Vol. 1, (CRC Press, Boca Raton, Fla., 1987), pp 39-90, describes encapsulation for controlled drug delivery. Heller, in: Hydrogels in Medicine and Pharmacy, N. A. Peppes ed., Vol. III, (CRC Press, Boca Raton, Fla., 1987), pp 137-149, describes bioerodible polymers.

Anderson et al., Contraception 13:375, (1976), and Miller et al., J. Biomed. Materials Res. 11:711, (1977) describe various properties of poly(dL-lactic acid).

Brine, U.S. Pat. No. 5,075,115 discloses controlled release formulations with lactic acid polymers and co-polymers.

Di Colo, Biomaterials 13:850-856 (1992) describes controlled drug release from hydrophobic polymers.

Olejnik, et al. U.S. Pat. No. 6,074,661 discloses an implantable device for treatment of an eye, wherein the device incorporates a retinoid for improving the biocompatibility of the device in eye tissue.

Wong, U.S. Pat. No. 6,699,493 discloses a method for reducing or preventing transplant rejection in the eye and intraocular implants for use therefore.

Other documents that are also relevant or otherwise helpful in understanding the present invention are U.S. patent application Ser. No. 09/693,008, filed on Jul. 5, 2000; Ser. No. 10/246,884, filed on Sep. 18, 2002; Ser. No. 10/327,018, filed on Dec. 20, 2002 and Ser. No. 10/340,237, filed on Jan. 9, 2003.

The entire disclosure of each of the documents cited herein is incorporated herein in its entirety by this reference.

### SUMMARY

The present invention provides new drug delivery systems, and methods of using such systems, for modified drug release into an eye, for example, to achieve one or more desired therapeutic effects. The present systems and methods advantageously provide for desired or substantially predetermined drug release rates, such as substantially consistent, for example, substantially constant, drug release rates, in the eye. Thus, the patient in whose eye the present drug delivery system implanted is benefited by having a substantially consistent level of active component within the eye for treatment of an ocular condition. For example, a patient with the present system implanted within an eye has a substantially consistent level of an active component available for consistent treatment of the eye over a relatively long period of time, for example, on the order of at least about 1 week or at least about 1 month or at least about 3 months or longer. Such consistent active component release rates facilitate obtaining successful treatment results.

Advantageously, the present delivery devices preferably are at least partially biodegradable so that removal of the device, after substantially complete active component release, is not required. The present drug delivery systems are relatively straightforward in structure, and can be relatively easily made and used to treat a wide variety of ocular conditions.

In one broad aspect of the invention, the drug delivery systems comprise one or more elements, hereinafter, sometimes interchangeably referred to as implants, sized and adapted for placement into an eye, for example into one of an anterior chamber of an eye and a posterior chamber of an eye. Such elements preferably include a core containing a therapeutic component, sometimes referred to elsewhere herein as an active component or a therapeutically active component, a first layer component located on a first side of the core, and a second layer component located on a substantially opposing second side of the core. Advantageously, the element may be structured such that a release rate of the therapeutic component from the core into the eye is reduced and/or is more consistent relative to a substantially identical element without the first and second layer components.

The present elements may have an outer periphery at least a portion of which is free of both the first layer component and the second layer component. For example, the elements may include a portion of the core, for example a peripheral surface of the core, that is exposed and not covered by either one of the first layer component or the second layer component.

In some embodiments of the invention, the first and second layer components substantially cover, conceal or surround an outer portion of the core. For example, each of the first and second layer components may have a surface that faces the core and has substantially the same shape as a surface of the core.

In accordance with the present invention, each of the first and second layer components may have an outer periphery having a suitable configuration or shape. Such shape may be selected to provide or obtain a desired release rate of the therapeutic component from the element. Examples, without limitation, of shapes of the outer peripheries of the first and second layer components include those having a cross section in a form selected from a circle, a rectangle, an oval, an ellipse, portions thereof, and combinations thereof.

In some embodiments of the invention, the first layer component includes a first polymeric material, and the second layer component includes a second polymeric material. For example, the first polymeric material and the second polymeric material may be the same material, or in the alternative, may be different materials, one from the other.

The first and second layer components may include no therapeutic component, for example, may be comprised of one or more substantially inactive agents, for example, a pure polymeric material. Alternately, one or both of the first and second layer components may include an active agent, for example, a concentration of the therapeutic component, for example, a reduced concentration of the therapeutic component relative to the concentration of the therapeutic component present in the core. The amount or concentration, if any, of the therapeutic component in one or both of the layer components may be selected to provide the desired release rate profile of the therapeutic component from the element.

In some embodiments of the invention, the implants have a gradient of drug concentrations, wherein the drug concentration is highest generally at a center of the implant, and the drug concentration decreases outwardly therefrom. The concentration gradient is controllable by appropriate selection of manufacturing parameters. For example, a relatively gradual gradient can be achieved through higher pressures and or temperatures during the compression of the implant. Similarly, the gradient can be effectively decreased or minimized by appropriate selection of manufacturing parameters, for example by decreasing pressure and/or temperature during the compression of the implants.

The core of the implants of the present invention can include a therapeutic component alone, or dispersed within an substantially inactive component. For example, the core may comprise an active, therapeutic drug combined with a polymeric material, for example a biodegradable polymeric material. The core compositions can vary according to the preferred drug release profile, the particular active agent used, the ocular condition being treated, and the medical history of the patient.

For example, in some embodiments of the present invention, the core of the element advantageously comprises a therapeutic component admixed with one or more matrix materials, for example, one or more polymeric materials, for example biodegradable polymeric materials. In these embodiments of the invention, the core may be structured to provide a controlled rate of release of the active agent therefrom upon erosion or degradation of an inactive, bioerodible material, for example polymeric material, present within the core. More specifically, the implant may be structured such that controlled release of the biodegradable polymer matrix may comprise at least about 10 percent, at least about 20 percent, at least about 30 percent, at least about 40 percent, at least about 50 percent, at least about 60 percent, at least about 70 percent, at least about 80 percent, at least about 90 percent of the core.

Biodegradable polymers which can be used include, but are not limited to, polymers made of monomers such as organic esters or ethers, which when degraded result in physiologically acceptable degradation products. Anhydrides, amides, orthoesters, or the like, by themselves or in combination with other monomers, may also be used. The polymers are generally condensation polymers. The polymers can be crosslinked or non-crosslinked. If crosslinked, they are usually not more than lightly crosslinked, and are less than 5% crosslinked, usually less than 1% crosslinked.

For the most part, besides carbon and hydrogen, the polymers will include oxygen and nitrogen, particularly oxygen. The oxygen may be present as oxy, e.g., hydroxy or ether, carbonyl, e.g., non-oxo-carbonyl, such as carboxylic acid ester, and the like. The nitrogen can be present as amide, cyano, and amino. An exemplary list of biodegradable polymers that can be used are described in Heller, “Biodegradable Polymers in Controlled Drug Delivery”, in: CRC Critical Reviews in Therapeutic Drug Carrier Systems, Vol. 1. (CRC Press, Boca Raton, Fla., 1987).

Of particular interest are polymers of hydroxyaliphatic carboxylic acids, either homo- or copolymers, and polysaccharides. Included among the polyesters of interest are homo- or copolymers of D-lactic acid, L-lactic acid, racemic lactic acid, glycolic acid, caprolactone, and combinations thereof. Copolymers of glycolic and lactic acid are of particular interest, where the rate of biodegradation is controlled by the ratio of glycolic to lactic acid. The percent of each monomer in poly(lactic-co-glycolic)acid (PLGA) copolymer may be 0-100%, about 15-85%, about 25-75%, or about 35-65%. In certain variations, 25/75 PLGA and/or 50/50 PLGA copolymers are used. In other variations, PLGA copolymers are used in conjunction with polylactide polymers.

Biodegradable polymer matrices that include mixtures of hydrophilic and hydrophobic ended PLGA may also be employed, and are useful in modulating polymer matrix degradation rates. Hydrophobic ended (also referred to as capped or end-capped) PLGA has an ester linkage hydrophobic in nature at the polymer terminus. Typical hydrophobic end groups include, but are not limited to alkyl esters and aromatic esters. Hydrophilic ended (also referred to as uncapped) PLGA has an end group hydrophilic in nature at the polymer terminus. PLGA with a hydrophilic end groups at the polymer terminus degrades faster than hydrophobic ended PLGA because it takes up water and undergoes hydrolysis at a faster rate (Tracy et al., Biomaterials 20:1057-1062 (1999)). Examples of suitable hydrophilic end groups that may be incorporated to enhance hydrolysis include, but are not limited to, carboxyl, hydroxyl, and polyethylene glycol. The specific end group will typically result from the initiator employed in the polymerization process. For example, if the initiator is water or carboxylic acid, the resulting end groups will be carboxyl and hydroxyl. Similarly, if the initiator is a monofunctional alcohol, the resulting end groups will be ester or hydroxyl.

The core composition may be monolithic, that is, having the therapeutic component substantially uniformly distributed throughout the core, for example, throughout the polymeric material present in the core, or the core may have encapsulated reservoirs for example, particles and/or other relatively concentrated forms, of therapeutic component interspersed throughout the core, for example, throughout the polymeric material in the core.

Therapeutic, active agents that may be used in the systems and methods of the present invention include, but are not limited to (either by itself or in combination with another active agent): ace-inhibitors, endogenous cytokines, agents that influence basement membrane, agents that influence the growth of endothelial cells, adrenergic agonists or blockers, cholinergic agonists or blockers, aldose reductase inhibitors, analgesics, anesthetics, antiallergics, anti-inflammatory agents, antihypertensives, pressors, antibacterials, antivirals, antifungals, antiprotozoals, anti-infectives, antitumor agents, antimetabolites, antiangiogenic agents, tyrosine kinase inhibitors, antibiotics such as aminoglycosides such as gentamycin, kanamycin, neomycin, and vancomycin; amphenicols such as chloramphenicol; cephalosporins, such as cefazolin HCl; penicillins such as ampicillin, penicillin, carbenicillin, oxycillin, methicillin; lincosamides such as lincomycin; polypeptide antibiotics such as polymixin and bacitracin; tetracyclines such as tetracycline; quinolones such as ciproflaxin, etc.; sulfonamides such as chloramine T; and sulfones such as sulfanilic acid as the hydrophilic entity, anti-viral drugs, e.g. acyclovir, gancyclovir, vidarabine, azidothymidine, dideoxyinosine, dideoxycytosine, dexamethasone, ciproflaxin, water soluble antibiotics, such as acyclovir, gancyclovir, vidarabine, azidothymidine, dideoxyinosine, dideoxycytosine; epinephrine; isoflurphate; adriamycin; bleomycin; mitomycin; ara-C; actinomycin D; scopolamine; and the like, analgesics, such as codeine, morphine, keterolac, naproxen, etc., an anesthetic, e.g. lidocaine; .beta.-adrenergic blocker or .beta.-adrenergic agonist, e.g. ephidrine, epinephrine, etc.; aldose reductase inhibitor, e.g. epalrestat, ponalrestat, sorbinil, tolrestat; antiallergic, e.g. cromolyn, beclomethasone, dexamethasone, and flunisolide; colchicine, anihelminthic agents, e.g. ivermectin and suramin sodium; antiamebic agents, e.g. chloroquine and chlortetracycline; and antifungal agents, e.g. amphotericin, etc., anti-angiogenesis compounds such as anecortave acetate, retinoids such as Tazarotene, anti-glaucoma agents, such as brimonidine (Alphagan and Alphagan P), acetozolamide, bimatoprost (Lumigan), timolol, timolol maleate, mebefunolol; memantine; alpha-2 adrenergic receptor agonists; 2ME2; anti-neoplastics, such as vinblastine, vincristine, interferons; alpha., beta. and .gamma., antimetabolites, such as folic acid analogs, purine analogs, and pyrimidine analogs; immunosuppressants such as azathiprine, cyclosporine and mizoribine; miotic agents, such as carbachol, mydriatic agents such as atropine, etc., protease inhibitors such as aprotinin, camostat, gabexate, vasodilators such as bradykinin, etc., and various growth factors, such epidermal growth factor, basic fibroblast growth factor, nerve growth factors, and the like.

In one embodiment of the invention, the active agent is methotrexate. In another embodiment, the active agent is a retinoic acid. In another embodiment, the active agent is an anti-inflammatory agent such as a nonsteroidal anti-inflammatory agent. Nonsteroidal anti-inflammatory agents that may be used include, but are not limited to, aspirin, diclofenac, flurbiprofen, ibuprofen, ketorolac, naproxen, and suprofen. In a further variation, the anti-inflammatory agent is a steroidal anti-inflammatory agent.

The steroidal anti-inflammatory agents that may be used in the systems of the present invention include, but are not limited to, 21-acetoxypregnenolone, alclometasone, algestone, amcinonide, beclomethasone, betamethasone, budesonide, chloroprednisone, clobetasol, clobetasone, clocortolone, cloprednol, corticosterone, cortisone, cortivazol, deflazacort, desonide, desoximetasone, dexamethasone, diflorasone, diflucortolone, difluprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, halopredone acetate, hydrocortamate, hydrocortisone, loteprednol etabonate, mazipredone, medrysone, meprednisone, methylprednisolone, mometasone furoate, paramethasone, prednicarbate, prednisolone, prednisolone 25-diethylamino-acetate, prednisolone sodium phosphate, prednisone, prednival, prednylidene, rimexolone, tixocortol, triamcinolone, triamcinolone acetonide, triamcinolone benetonide, triamcinolone hexacetonide, and any of their derivatives.

In one aspect of the invention, cortisone, dexamethasone, fluocinolone, hydrocortisone, methylprednisolone, prednisolone, prednisone, and triamcinolone, and their derivatives, are preferred steroidal anti-inflammatory agents. In another aspect of the invention, the steroidal anti-inflammatory agent is dexamethasone. In another aspect of the invention, the biodegradable implant includes a combination of two or more steroidal anti-inflammatory agents.

The active agent can comprise from about 10% to about 90% by weight of the element or implant. In one variation, the agent is from about 40% to about 80% by weight of the implant. In a preferred variation, the agent comprises about 60% by weight of the implant. In a more preferred embodiment of the present invention, the agent can comprise about 50% by weight of the implant.

Other agents may be employed in the formulation for a variety of purposes. For example, buffering agents and preservatives may be employed. Preservatives which may be used include, but are not limited to, sodium bisulfite, sodium bisulfate, sodium thiosulfate, benzalkonium chloride, chlorobutanol, thimerosal, phenylmercuric acetate, phenylmercuric nitrate, methylparaben, polyvinyl alcohol and phenylethyl alcohol. Examples of buffering agents that may be employed include, but are not limited to, sodium carbonate, sodium borate, sodium phosphate, sodium acetate, sodium bicarbonate, and the like, as approved by the FDA for the desired route of administration. Electrolytes such as sodium chloride and potassium chloride may also be included in the formulation.

The implants in accordance with the present invention can also include hydrophilic or hydrophobic compounds that accelerate or retard release of the active agent. Additionally, release modulators such as those described in U.S. Pat. No. 5,869,079 can be included in the implants. The amount of release modulator employed will be dependent on the desired release profile, the activity of the modulator, and on the release profile of the glucocorticoid in the absence of modulator. Where the buffering agent or release enhancer or modulator is hydrophilic, it may also act as a release accelerator. Hydrophilic additives act to increase the release rates through faster dissolution of the material surrounding the drug particles, which increases the surface area of the drug exposed, thereby increasing the rate of drug diffusion. Similarly, a hydrophobic buffering agent or enhancer or modulator can dissolve more slowly, slowing the exposure of drug particles, and thereby slowing the rate of drug diffusion.

An element or implant within the scope of the present invention can be formulated with particles of an active agent dispersed within a biodegradable polymer matrix. Without being bound by theory, it is believed that the release of the active agent can be achieved by erosion of the biodegradable polymer matrix and by diffusion of the particulate agent into an ocular fluid, for example, vitreal fluid, with contemporaneous or subsequent dissolution of the polymer matrix and release of the active agent.

Factors which influence the release kinetics of active agent from the implant can include such characteristics as the size and shape of the implant, the size of the active agent particles, the solubility of the active agent, the ratio of active agent to polymer(s), the method of manufacture, the surface area exposed, and the erosion rate of the polymer(s). The release kinetics achieved by this form of active agent release are different than that achieved through formulations which release active agents through polymer swelling, such as with crosslinked hydrogels. In that case, the active agent is not released through polymer erosion, but through polymer swelling and drug diffusion, which releases agent as liquid diffuses through the pathways exposed.

The release rate of the active agent can depend at least in part on the rate of degradation of the polymeric component or components making up the biodegradable polymer matrix. For example, condensation polymers may be degraded by hydrolysis (among other mechanisms) and therefore any change in the composition of the implant that enhances water uptake by the implant will likely increase the rate of hydrolysis, thereby increasing the rate of polymer degradation and erosion, and thus increasing the rate of active agent release.

The release kinetics of the implants of the present invention can be dependent in part on the surface area of the implants. A larger surface area, for example a surface area of an exposed portion of the core that is not covered by the first layer component or the second layer component, exposes more of the core composition to ocular fluid, causing faster erosion of the polymer matrix of the core and faster dissolution of the active agent particles in the fluid. Therefore, the size and shape of the implant may also be used to control the rate of release, period of treatment, and active agent concentration at the site of implantation. At equal active agent loads, larger implants will deliver a proportionately larger dose, but depending on the surface to mass ratio, may possess a slower release rate.

The implants in accordance with the present invention are typically solid, and may be formed as layered particles, sheets, patches, plaques, films, discs, fibers, rods, and the like, or may be of any size or shape compatible with the selected site of implantation, as long as the implants have the desired release kinetics and deliver an amount of active agent that is therapeutic for the intended medical condition of the eye. The upper limit for the implant size will be determined by factors such as the desired release kinetics, toleration for the implant at the site of implantation, size limitations on insertion, and ease of handling. For example, the vitreous chamber is able to accommodate relatively large rod-shaped implants, generally having diameters of about 0.05 mm to 3 mm and a length of about 0.5 to about 10 mm. In one variation, the rods have diameters of about 0.1 mm to about 1 mm. In another variation, the rods have diameters of about 0.3 mm to about 0.75 mm. In yet a further variation, other implants having variable geometries but approximately similar volumes may also be used.

The proportions of active agent, polymer, and any other modifiers may be empirically determined by formulating several implants with varying proportions. A USP approved method for dissolution or release test can be used to measure the rate of release (USP 23; NF 18 (1995) pp. 1790-1798). For example, using the infinite sink method, a weighed sample of the drug delivery device is added to a measured volume of a solution containing 0.9% NaCl in water, where the solution volume will be such that the drug concentration is after release is less than 20%, and preferably less than 5%, of saturation. The mixture is maintained at 37° C. and stirred slowly to ensure drug diffusion after bioerosion. The appearance of the dissolved drug as a function of time may be followed by various methods known in the art, such as spectrophotometrically, HPLC, mass spectroscopy, etc.

At least a portion of the element preferably is biodegradable or bioerodible. For example, at least one of the first polymeric material and the second polymeric material preferably is at least partially or substantially completely biodegradable or bioerodible.

In the present context, a biodegradable or bioerodible material is one which degrades into physiologically acceptable degradation products under physiological conditions in the eye, or erodes into physiologically acceptable materials under physiological conditions in the eye. In addition, the polymeric material present in the core may be and preferably is, biodegradable or bioerodible.

In some embodiments of the invention, the element is structured such that upon being implanted into an eye, for example into a vitreous of an eye, each one of the first layer component, the second layer component and the core biodegrades or bioerodes at a substantially uniform rate and in a substantially uniform manner in relation to each other one of the first layer component, second layer component and core. In other words, in some embodiments of the invention, the element is structured to degrade or erode in the ocular environment at a rate and in a manner such that the configuration or shape of the element remains substantially consistent throughout the treatment period.

In another aspect of the present invention the element may be substantially solid or nonporous in that there are no substantial pores or openings within one or more of the outer surfaces thereof. In this aspect of the invention, release of the therapeutic component may be effected by exposure of a peripheral surface of the core which substantially circumscribes the element between peripheral surfaces of the first layer component and the second layer component.

In another aspect of the invention, the element includes an aperture defined within at least one of the first layer component, the second layer component, and the core. For example, in some embodiments of the invention, the core containing therapeutic agents may be substantially sealed within the first and second layer components comprising substantially no active agents, for example comprising substantially pure polymeric materials. Release of the therapeutic agent into the eye may be effected by means of one or more apertures, openings, or holes defined within the at least one of the first and second layer components.

In one particularly advantageous embodiment, the element includes a generally centrally located aperture, for example, through all of the first layer component, core and second layer component. This embodiment of the invention provides a biodegradable element that has a relatively more consistent or more constant release rate of the therapeutic component into the eye over time relative to a substantially identical element without such generally centrally located aperture.

The systems of the invention may comprise a plurality of the elements as described and shown herein.

In some embodiments of the invention, the therapeutic component of the core is a drug that is substantially insoluble in the eye, and the core polymeric material is structured to have a faster degradation rate in the eye than at least one of, and preferably both of, the first and second polymeric materials. In other embodiments of the invention, the therapeutic component is a drug that is substantially soluble in the eye, and at least two of, and preferably all of, the core polymeric material, the first polymeric material, have substantially the same degradation rate.

The core may include a single therapeutic agent or a plurality of different therapeutic gents depending upon the nature of the condition or conditions of the eye being treated. The site of implantation of the element of the invention can vary depending upon the ocular condition being treated and the desired course of treatment.

For example, the present systems may be structured for treatment of an inflammation mediated condition, for example, uveitis. In this case, the therapeutic component may comprise an anti-inflammatory agent, for example, and is preferably implanted proximal to the uveal structures.

For example, the present systems may be structured for treatment of glaucoma. The element may be structured to provided sustained release of one or more neuroprotective agents that protect cells from excitotoxic damage. The element may be structured to be effective in delivering one or more beta-blockers, for example Timolol Maleate, to the eye on a substantially consistent basis. Other agents include N-methyl-D-aspartate (NMDA) antagonists, cytokines, and neurotrophic factors, preferably delivered intravitreally.

For example, the present systems may be structured for treatment of diabetic retinopathy. The therapeutic component may comprise one or more anti-angiogenic agents and/or one or more neurotropic agents, and may be structured to be implanted within the vitreous.

The present systems may be structured for treating age-related macular degeneration. For example, elements are provided for delivery of one or more neurotrophic factors intraocularly, preferably to the vitreous, and/or one or more anti-angiogenic factors intraocularly or periocularly, preferably periocularly, most preferably to the sub-Tenon's region.

The present invention also provides methods of treating an eye, for example including the step of placing the drug delivery system described herein into an eye.

Each and every feature described herein, and each and every combination of two or more of such features, is included within the scope of the present invention provided that the features included in such a combination are not mutually inconsistent.

Additional aspects and advantages of the present invention are set forth in the following description and claims, particularly when considered in conjunction with the accompanying drawings in which like parts bear like reference numerals.

### DRAWINGS

FIG. 1 shows a perspective view of a drug delivery system element, or implant, in accordance with the present invention.

FIGS. 1a, 1b and 1c are schematic illustrations of alternate top or sideview surface shapes of the elements shown in FIG. 1.

FIG. 2 shows a drug release profile over time of the element shown in FIG. 1.

FIG. 3 shows a side view of another drug delivery system element in accordance with the present invention.

FIG. 3a is a cross sectional view of the element shown in FIG. 3.

FIGS. 4 and 5 are simplified schematic illustrations of sequential steps used in preparation of a drug delivery system element in accordance with the present invention.

FIG. 6 shows drug release profiles over time of the element shown in FIG. 3.

FIG. 7 shows a top view of an additional drug delivery system element in accordance with the present invention.

FIG. 7a is a cross sectional view of the element shown in FIG. 7, taken along line 7a-7a.

FIG. 8 shows a core layer of the element shown in FIG. 7, showing initial drug release from the outer and inner peripheries thereof.

FIG. 8a shows drug release from the core layer shown in FIG. 8 after the element has been in the eye for a period of time.

FIG. 9 shows a cross-sectional view of an eye.

### DESCRIPTION

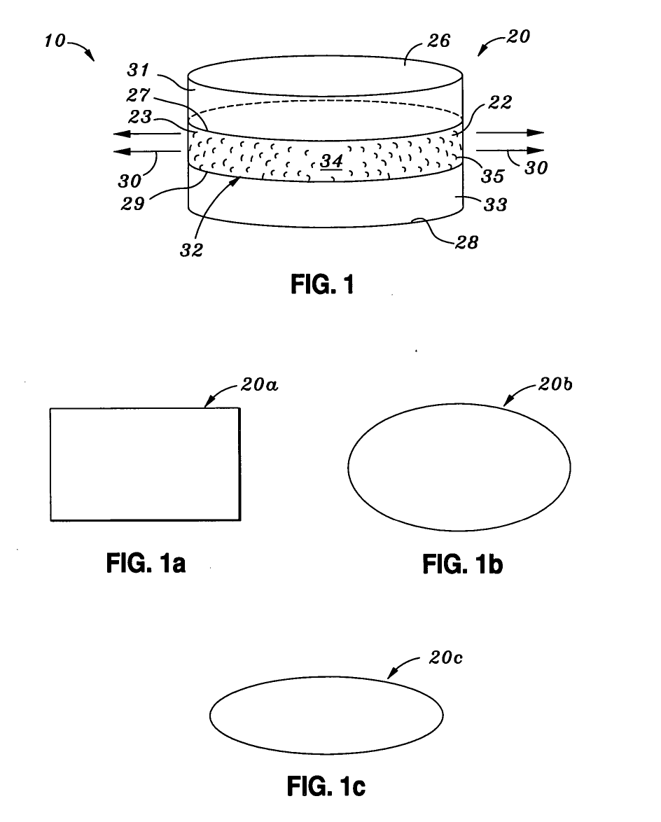

Turning now to FIG. 1, a drug delivery system in accordance with the present invention is shown generally at 10.

The present drug delivery systems of the present invention are generally directed to controlled release drug delivery system implants and methods for the treatment of ocular conditions, such as an anterior ocular condition, a posterior ocular condition, or an ocular condition which can be characterized as both an anterior ocular condition and a posterior ocular condition.

As used herein, and as generally understood by those of skill in the art, an ocular condition can include a disease, aliment or condition which affects or involves the eye or one of the parts or regions of the eye. Broadly speaking, the eye includes the eyeball and the tissues and fluids which constitute the eyeball, the periocular muscles (such as the oblique and rectus muscles) and the portion of the optic nerve which is within or adjacent to the eyeball.

An anterior ocular condition is a disease, ailment or condition which affects or which involves an anterior (i.e. front of the eye) ocular region or site, such as a periocular muscle, an eye lid or an eye ball tissue or fluid which is located anterior to the posterior wall of the lens capsule or ciliary muscles. Thus, an anterior ocular condition primarily affects or involves, the conjunctiva, the cornea, the conjunctiva, the anterior chamber, the iris, the posterior chamber (behind the retina but in front of the posterior wall of the lens capsule), the lens or the lens capsule and blood vessels and nerve which vascularize or innervate an anterior ocular region or site. An anterior ocular condition can include a disease, ailment or condition, such as for example, aphakia; pseudophakia; astigmatism; blepharospasm; cataract; conjunctival diseases; conjunctivitis; corneal diseases; corneal ulcer; dry eye syndromes; eyelid diseases; lacrimal apparatus diseases; lacrimal duct obstruction; myopia; presbyopia; pupil disorders; refractive disorders and strabismus. Glaucoma can also be considered to be an anterior ocular condition because a clinical goal of glaucoma treatment can be to reduce a hypertension of aqueous fluid in the anterior chamber of the eye.

A posterior ocular condition is a disease, ailment or condition which primarily affects or involves a posterior ocular region or site such as choroid or sclera (in a position posterior to a plane through the posterior wall of the lens capsule), vitreous, vitreous chamber, retina, optic nerve (i.e. the optic disc), and blood vessels and nerves which vascularize or innervate a posterior ocular region or site. Thus, a posterior ocular condition can include a disease, ailment or condition, such as for example, macular degeneration (such as non-exudative age related macular degeneration and exudative age related macular degeneration); choroidal neovascularization; acute macular neuroretinopathy; macular edema (such as cystoid macular edema and diabetic macular edema); Behcet's disease, retinal disorders, diabetic retinopathy (including proliferative diabetic retinopathy); retinal arterial occlusive disease; central retinal vein occlusion; uveitic retinal disease; retinal detachment; ocular trauma which affects a posterior ocular site or location; a posterior ocular condition caused by or influenced by an ocular laser treatment; posterior ocular conditions caused by or influenced by a photodynamic therapy; photocoagulation; radiation retinopathy; epiretinal membrane disorders; branch retinal vein occlusion; anterior ischemic optic neuropathy; non-retinopathy diabetic retinal dysfunction, retinitis pigmentosa and glaucoma. Glaucoma can be considered a posterior ocular condition because the therapeutic goal is to prevent the loss of or reduce the occurrence of loss of vision due to damage to or loss of retinal cells or optic nerve cells (i.e. neuroprotection).

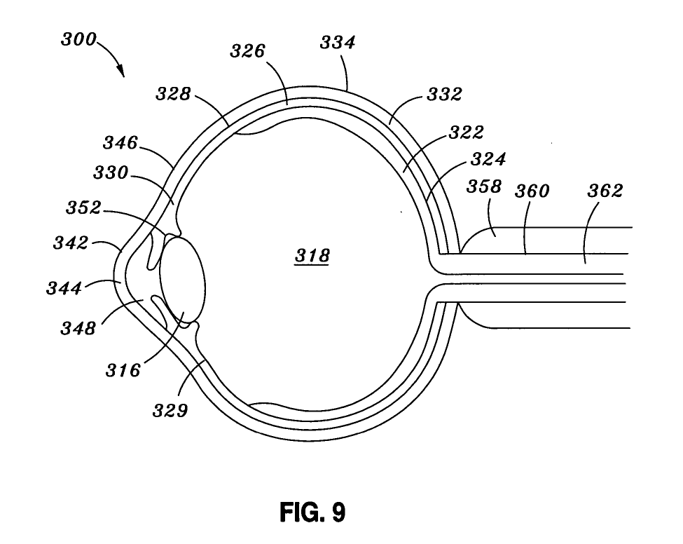

The system 10, in accordance with this particular embodiment of the invention, generally comprises an element 20 (hereinafter sometimes referred to as an implant), sized and adapted for placement into an eye, for example a human or animal eye, such as eye 300 shown in FIG. 9. The element 20 includes a core 22 containing an therapeutic component 23, a first layer component 26 located on a first side of the core 22, and a second layer component 28 located on a substantially opposing second side of the core 22. Advantageously, the element 20 may be structured such that a release, for example a rate of release over time, of the therapeutic component 23 from the core 22 (such release being illustrated by arrows 30) into the eye is modified or reduced relative to a substantially identical element without one or both of the first layer component 26 and the second layer component 28. Each of the first and second layer components 26, 28 preferably has a surface 27, 29 facing the core which is substantially the same shape as the surface of the core 22 facing each of the first and second layer components. Therefore, as shown, the element 20 may be in the form of a multilayered component having two or more layers or laminates having different compositions and/or different concentrations or ratios of active to inactive agents within each layer or laminate.

Release of the therapeutic component 23 into the eye generally takes place at or from portions of the core 22 that are directly exposed to the physiological environment of the eye when the element 20 is implanted therein. For example, the element 20 may be structured such that the core 22 includes an outer periphery 32 at least a portion of which is free of the first layer component 26 and the second layer component 28. More specifically, in the embodiment of the invention shown in FIG. 1, the core 22 of the element 20 has an outer peripheral surface 34 which is free of the first layer component 26 and the second layer component 28.

Preferably, the first layer component 26 includes a first polymeric material 31 and the second layer component 28 includes a second polymeric material 33. In some embodiments of the invention, the first polymeric material 31 and the second polymeric material 33 are the same polymeric material, that is are polymeric materials having substantially the same chemical composition. In addition, preferably the core 22 includes the therapeutic component 23 combined with a core polymeric material 35. The polymeric materials in the first layer component 26, second layer component 28 and the core 22 may all comprise the same polymeric material, or may comprise different polymeric materials, in accordance with different embodiments of the invention.

The first and second layer components 26, 28 may include no therapeutic component, for example, may be comprised of substantially pure polymeric material. Alternately, one or both of the first and second layer components 26, 28 may include a physiologically active agent, for example, a concentration of a therapeutic component, for example, a reduced concentration of the therapeutic component 23 relative to the concentration of the therapeutic component 23 present in the core 22.

The amount or concentration, if any, of the therapeutic component present in one or both of the layer components 26, 28 may be selected to provide, or to at least assist in providing, the desired release rate profile of that therapeutic component from the element 20.

Suitable polymeric materials or compositions for use in the layer components and cores of the present invention include those materials which are compatible, that is biocompatible, with the eye so as to cause no substantial interference with the functioning or physiology of the eye. Such materials preferably are at least partially and more preferably substantially completely biodegradable or bioerodible. The selection of the biodegradable polymer used can vary with the desired release kinetics, patient tolerance, the nature of the disease to be treated, and the like. Examples of useful polymeric materials include, without limitation, such materials derived from and/or including organic esters and organic ethers, which when degraded result in physiologically acceptable degradation products, including the monomers. Also, polymeric materials derived from and/or including, anhydrides, amides, orthoesters and the like, by themselves or in combination with other monomers, may also find use. The polymeric materials may be addition or condensation polymers, advantageously condensation polymers. The polymeric materials may be cross-linked or non-cross-linked, for example not more than lightly cross-linked, such as less than about 5%, or less than about 1% of the polymeric material being cross-linked. For the most part, besides carbon and hydrogen, the polymers will include at least one of oxygen and nitrogen, advantageously oxygen. The oxygen may be present as oxy, e.g. hydroxy or ether, carbonyl, e.g. non-oxo-carbonyl, such as carboxylic acid ester, and the like. The nitrogen may be present as amide, cyano and amino. The polymers set forth in Heller, Biodegradable Polymers in Controlled Drug Delivery, In: CRC Critical Reviews in Therapeutic Drug Carrier Systems, Vol. 1, CRC Press, Boca Raton, Fla. 1987, pp 39-90, which describes encapsulation for controlled drug delivery, may find use in the present invention, and that disclosure is specifically incorporated herein by reference.

Of additional interest are polymers of hydroxyaliphatic carboxylic acids, either homopolymers or copolymers, and polysaccharides. Included among the polyesters of interest are polymers of D-lactic acid, L-lactic acid, racemic lactic acid, glycolic acid, polycaprolactone, and combinations thereof. Generally, by employing the L-lactate, a slowly eroding polymer or polymeric material is achieved, while erosion is substantially enhanced with the lactate racemate.

Among the useful polysaccharides are, without limitation, calcium alginate, and functionalized celluloses, particularly carboxymethylecellulose esters characterized by being water insoluble, a molecular weight of about 5 kD to 500 kD, etc.

Other polymers of interest include, without limitation, polyvinyl alcohol, polyesters, polyethers and combinations thereof which are biocompatible and may be biodegradable and/or bioerodible.

Some preferred characteristics of the polymers or polymeric materials for use in the present invention may include biocompatibility, compatibility with the therapeutic component, ease of use of the polymer in making the drug delivery systems of the present invention, a half life in the physiological environment of at least about 6 hours, preferably greater than about one day, not significantly increasing the viscosity of the vitreous, and water insolubility.

The biodegradable polymeric materials which are included form the first layer component 26, second layer component 28 and/or the core 22 are desirably subject to enzymatic or hydrolytic instability. Water soluble polymers may be cross-linked with hydrolytic or biodegradable unstable cross-links to provide useful water insoluble polymers. The degree of stability can be varied widely, depending upon the choice of monomer, whether a homopolymer or copolymer is employed, employing mixtures of polymers, where the polymers may be employed as varying layers or mixed.

The element 20 advantageously is structured to have a lifetime at least equal to the desired period of therapeutic component administration in the eye, and may have lifetimes of about 5 to about 10 times the desired period of administration. The period of administration may be at least about 3 days, at least about 7 days, at least about 15 days, at least about 20 days, at least about 30 days or longer.

The therapeutic component useful in the present invention may include any suitable pharmacologically active agent or therapeutic agent for which sustained, modified, extended, delayed, or otherwise controlled release in the eye, is desirable. Advantageously, the therapeutic component is preferably sufficiently soluble in the vitreous of the eye such that it will be present at a pharmacologically or otherwise therapeutically effective dose. Pharmacologic or therapeutic agents which may find use in the present systems, include, without limitation, those disclosed in U.S. Pat. No. 4,474,451, columns 4-6 and U.S. Pat. No. 4,327,725, columns 7-8, which disclosures are incorporated herein by reference.

Pharmacological or therapeutic agents of interest include hydrocortisone (5-20 mcg/l as plasma level), gentamycin (6-10 mcg/ml in serum), 5-fluorouracil (about 0.30 mg/kg body weight in serum), sorbinil, IL-2, TNF, Phakan-a (a component of glutathione), thioloa-thiopronin, Bendazac, acetylsalicylic acid, trifluorothymidine, interferon (alpha., beta. and gamma.), immune modulators, e.g. lymphokines, monokines, and growth factors, etc.

Pharmacological or therapeutic agents of particular interest include, without limitation, anti-glaucoma drugs, such as the beta-blockers, such as timolol maleate, betaxolol and metipranolol; mitotics, such as pilocarpine, acetylcholine chloride, isofluorophate, demacarium bromide, echothiophate iodide, phospholine iodide, carbachol, and physostigimine; epinephrine and salts, such as dipivefrin hydrochloride; and dichlorphenamide, acetazolamide and methazolamide; anti-cataract and anti-diabetic retinopathy drugs, such as aldose reductase inhibitors, such as tolrestat, lisinopril, enalapril, and statil; thiol cross-linking drugs other than those considered previously; anti-cancer drugs, such as retinoic acid, methotrexate, adriamycin, bleomycin, triamcinoline, mitomycin, cis-platinum, vincristine, vinblastine, actinomycin-D, ara-c, bisantrene, CCNU, activated cytoxan, DTIC, HMM, melphalan, mithramycin, procarbazine, VM26, VP16, and tamoxifen; immune modulators, other than those indicated previously; anti-clotting agents, such as tissue plasminogen activator, urokinase, and streptokinase; anti-tissue damage agents, such as superoxide dismutase; proteins and nucleic acids, such as mono- and polyclonal antibodies, enzymes, protein hormones and genes, gene fragments and plasmids; steroids, particularly anti-inflammatory or anti-fibrous drugs, such as cortisone, hydrocortisone, prednisolone, prednisone, dexamethasone, progesterone-like compounds, medrysone (HMS) and fluorometholone; non-steroidal anti-inflammatory drugs, such as ketorolac tromethamine, dichlofenac sodium and suprofen; antibiotics, such as loridine (cephaloridine), chloramphenicol, clindamycin, amikacin, tobramycin, methicillin, lincomycin, oxycillin, penicillin, amphotericin B, polymyxin B, cephalosporin family, ampicillin, bacitracin, carbenicillin, cepholothin, colistin, erythromycin, streptomycin, neomycin, sulfacetamide, vancomycin, silver nitrate, sulfisoxazole diolamine, and tetracycline; other antipathogens, including anti-viral agents, such as idoxuridine, trifluorouridine, vidarabine (adenine arabinoside), acyclovir (acycloguanosine), pyrimethamine, trisulfapyrimidine-2, clindamycin, nystatin, flucytosine, natamycin, miconazole and piperazie derivatives, e.g. diethylcarbamazine; cycloplegic and mydriatic agents, such as atropine, cyclogel, scopolamine, homatropine and mydriacyl; and the like and mixtures thereof.

Other agents useful in the systems of the present invention include, without limitation, anticholinergics, anticoagulants, antifibrinolytic agents, antihistamines, antimalarials, antitoxins, chelating agents, hormones, immunosuppressives, thrombolytic agents, vitamins, salts, desensitizing agents, prostaglandins, amino acids, metabolites, antiallergenics, and the like and mixtures thereof.

In a particularly advantageous embodiment of the invention, systems suitable for treating oculan neuropathies, such as various forms of glaucoma, are provided. In such embodiments, the present invention provides an effective system for delivering highly soluble drugs, for example timolol, timolol maleate, derivatives thereof and combinations thereof, to an eye on a sustained or extended release basis without exhibiting any significant initial burst effect of the drug into the eye.

In another particularly advantageous embodiment of the invention, systems suitable for treating inflammation-mediated conditions of the eye are provided. The term “inflammation-mediated condition of the eye” is meant to include any condition of the eye which may benefit from treatment with an anti-inflammatory agent, and is meant to include, but is not limited to, uveitis, macular edema, acute macular degeneration, retinal detachment, ocular tumors, fungal or viral infections, multifocal choroiditis, diabetic uveitis, proliferative vitreoretinopathy (PVR), sympathetic opthalmia, Vogt Koyanagi-Harada (VKH) syndrome, histoplasmosis, and uveal diffusion.

For example, the systems may comprise an element, such as element 20, structured for being implanted into the vitreous of the eye wherein the core 22 comprises a steroidal anti-inflammatory agent, for example but not limited to, dexamethasone, and a bioerodible polymer. The element 20 preferably delivers the agent to the vitreous in an amount sufficient to reach a concentration equivalent to at least about 0.05 μg/ml dexamethasone within about 48 hours and maintains a concentration equivalent to at least about 0.03 μg/ml dexamethasone for at least about three weeks. In another embodiment of the invention, the element 20 preferably delivers the agent to the vitreous in an amount sufficient to reach a concentration equivalent to at least about 0.2 μg/ml dexamethasone within about 6 hours and maintains a concentration equivalent to at least about 0.01 μg/ml dexamethasone for at least about three weeks.

“A concentration equivalent to dexamethasone” means the concentration of a steroidal anti-inflammatory agent necessary to have approximately the same efficacy in vivo as a particular dose of dexamethasone. For example, hydrocortisone is approximately twentyfivefold less potent than dexamethasone, and thus a 25 mg dose of hydrocortisone would be equivalent to a 1 mg dose of dexamethasone. One of ordinary skill in the art would be able to determine the concentration equivalent to dexamethasone for a particular steroidal anti-inflammatory agent from one of several standard tests known in the art. Relative potencies of selected corticosteroids may be found, for example, in Gilman, A. G., et al., eds. (1990). Goodman and Gilman's: The Pharmacological Basis of Therapeutics. 8th Edition, Pergamon Press: New York, p. 1447, which is incorporated herein by this specific reference.

In other embodiments, the implant or element 20 delivers the agent to the vitreous in an amount sufficient to reach a concentration equivalent to at least about 0.3 μg/ml, or at least about 0.5 μg/ml, or at least about 0.75 μg/ml, or at least about 1.0 μg/ml, or at least about 2.0 μg/ml dexamethasone within about 4 hours, or within about 6 hours, or within about 8 hours, or within about 10 hours, or within about 24 hours.

A concentration equivalent to at least about 0.01 μg/ml, or at least about 0.02 μg/ml, or at least about 0.03 μg/ml, or at least about 0.05 μg/ml, or at least about 0.07 μg/ml dexamethasone may be maintained for an extended period of time (e.g., at least about three weeks or longer). The preferred concentration levels of therapeutic component or drug in the vitreous may vary according to the inflammatory mediated condition being treated. For example, for treating uveitis, a concentration equivalent of at least about 0.01 to 0.1 μg/ml dexamethasone is preferred.

In one embodiment, the concentration or therapeutic component is maintained for least about four weeks. In other embodiments, the concentration is maintained for at least about five weeks, or at least about six weeks, or at least about seven weeks, or at least about eight weeks, or at least about nine weeks, or at least about 10 weeks, or at least about 12 weeks or longer. The preferred duration of therapeutic component or drug release may be determined by the inflammatory mediated condition being treated. For treating uveitis, a drug release duration of at least about three weeks is preferable, more preferably at least about four weeks. In one embodiment, more than one implant or element 20 may be sequentially implanted into the vitreous in order to maintain therapeutic component or drug concentrations for even longer periods.

The implants or elements 20 of the present invention may be inserted into the eye, for example the vitreous chamber of the eye, by a variety of methods, including placement by forceps or by trocar following making a 2-3 mm incision in the sciera. The method of placement may influence the therapeutic component or drug release kinetics. For example, implanting the element 20 with a trocar may result in placement of the element 20 deeper within the vitreous than placement by forceps, which may result in the implant being closer to the edge of the vitreous. The location of the implanted element 20 may influence the concentration gradients of therapeutic component or drug surrounding the element, and thus influence the release rates (e.g., an element placed closer to the edge of the vitreous will result in a slower release rate).

The formulation of the implants in accordance with the present invention may vary according to the desired therapeutic component release profile, the particular therapeutic component used, the condition being treated, and the medical history of the patient.

In some embodiments of the invention, the core 22 of the element 20 is formulated with particles of a steroidal anti-inflammatory agent entrapped within a bioerodible polymer matrix, for example a polylactic acid polyglycolic acid (PLGA) copolymer. After implantation of the element 20 in the eye, release of the agent into the eye is achieved by erosion of core 22 at the exposed surface 34 thereof and erosion of the first layer component 26 and the second layer component 28.

In this embodiment of the invention, the first layer component 26 and/or the second layer component 28 may comprise a pure polymeric material or a polymeric material mixed with an active agent, for example a polymeric material mixed with a lower concentration of the steroidal anti-inflammatory agent relative to a concentration of the steroidal anti-inflammatory agent in the core 22. The erosion and release of the agent from the core 22 is achieved by exposure of previously entrapped agent particles to the vitreous, and subsequent dissolution and release of agent.

Some of the parameters which determine the release kinetics from the element 20 include the size of the therapeutic component or drug particles entrapped in the core 22, the water solubility of the therapeutic component or drug, the ratio of therapeutic component or drug to polymer, the method of manufacture, the amount of surface area of the core 22 exposed, the erosion rate of the polymer present in the core 22, and the thickness and composition of the first layer component 26 and the second layer component 28.

Preferably, the steroidal anti-inflammatory agent is selected from the group consisting of 21-acetoxypregnenolone, alclometasone, algestone, amcinonide, beclomethasone, betamethasone, budesonide, chloroprednisone, clobetasol, clobetasone, clocortolone, cloprednol, corticosterone, cortisone, cortivazol, deflazacort, desonide, desoximetasone, dexamethasone, diflorasone, diflucortolone, difluprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, halopredone acetate, hydrocortamate, hydrocortisone, loteprednol etabonate, mazipredone, medrysone, meprednisone, methylprednisolone, mometasone furoate, paramethasone, prednicarbate, prednisolone, prednisolone 25-diethylamino-acetate, prednisolone sodium phosphate, prednisone, prednival, prednylidene, rimexolone, tixocortol, triamcinolone, triamcinolone acetonide, triamcinolone benetonide, triamcinolone hexacetonide and the like and mixtures thereof. In a preferred embodiment, the steroidal anti-inflammatory agent is selected from the group consisting of cortisone, dexamethasone, hydrocortisone, methylprednisolone, prednisolone, prednisone, triamcinolone and the like and mixtures thereof. In a more preferred embodiment, the steroidal anti-inflammatory agent is dexamethasone. In another embodiment, the bioerodible implant comprises more than one steroidal anti-inflammatory agent.

The amount or concentrations of therapeutic component 23 employed in the core 22 will vary depending on the effective dosage required and rate of release.

For embodiments of the invention employing steroidal anti-inflammatory agents in the core, the polymers in the core and/or the first and second layer components may comprise, for example, polymers of hydroxyaliphatic carboxylic acids, either homo- or copolymers, and polysaccharides. Included among the polyesters of interest are polymers of D-lactic acid, L-lactic acid, racemic lactic acid, glycolic acid, polycaprolactone, and combinations thereof. By employing the L-lactate or D-lactate, a slowly biodegrading polymer is achieved, while degradation is substantially enhanced with the racemate.

Copolymers of glycolic and lactic acid are of particular interest, where the rate of biodegradation is controlled by the ratio of glycolic to lactic acid. The % of polylactic acid in the polylactic acid polyglycolic acid (PLGA) copolymer can be 0-100%, preferably about 15-85%, more preferably about 35-65%. In a particularly preferred embodiment, a 50/50 PLGA copolymer is used. The most rapidly degraded copolymer has roughly equal amounts of glycolic and lactic acid, where either homopolymer is more resistant to degradation. The ratio of glycolic acid to lactic acid will also affect the brittleness of in the implant, where a more flexible implant is desirable for larger geometries.

Other agents may be employed in the core 22 and/or first layer component 26 and/or second layer component 28 for a variety of purposes. In addition to the therapeutic component 23, effective amounts of buffering agents, preservatives and the like may be employed. Suitable water soluble preservatives include sodium bisulfite, sodium thiosulfate, ascorbate, benzalkonium chloride, chlorobutanol, thimerosal, phenylmercuric borate, parabens, benzyl alcohol, phenylethanol and the like and mixtures thereof. These agents may be present in amounts of from 0.001 to about 5% by weight and preferably 0.01 to about 2% by weight. Suitable water soluble buffering agents include, without limitation, alkali and alkaline earth carbonates, phosphates, bicarbonates, citrates, borates, acetates, succinates and the like, such as sodium phosphate, citrate, borate, acetate, bicarbonate, carbonate and the like. These agents advantageously present in amounts sufficient to maintain a pH of the system of between about 2 to about 9 and more preferably about 4 to about 8. As such the buffering agent may be as much as about 5% by weight of the total implant or element 20. Selection of an effective size and shape of the element 20 can be used to control the rate of release, period of treatment and drug concentration in the eye.

The element 20 in accordance with the present invention, may be of any suitable size, depending upon such factors as the type of condition being treated, the amount of therapeutic agent necessary for treatment of the condition, the desired length of the treatment, and the mode of administering the treatment (e.g. whether implantation is accomplished by injection with a needle, surgical implantation, forceps, trocar, or the like).

For example, the element 20 may have a size of between about 5 μm and about 2 mm, or between about 10 μm and about 1 mm for administration with a needle, greater than 1 mm, or greater than 2 mm, for administration by surgical implantation.

In some situations, the system 10 of the invention comprises a plurality of such elements 20 having the same or different size and/or shape, each employing the same or different therapeutic agent, and the same or different release rates and/or delayed release rates. For example, 2, 3, 4 or more elements in accordance with the present invention may be utilized. In this way, in a single administration a course of drug treatment may be achieved, where the pattern of release may be greatly varied. For example, a biphasic or triphasic release profile may be achieved with a single administration of a plurality of elements.

If the therapeutic component selected is an insoluble drug, then preferably the polymeric material mixed therewith in the core 22 preferably is selected to have a relatively faster rate of degradation than the polymeric compositions used in the first and second layer components 26, 28. In one embodiment, the first and second layer components 26, 28 are structured to have a smaller thickness than the core 22, for example, when the degradation rates of the first and second layer components are relatively low compared to the therapeutic component release rate from the core.

In addition, if the therapeutic component selected is a soluble drug, then polymeric materials having the same or closely similar rates of degradation can advantageously be used in the core 22, first layer component 26 and second layer component 28.

In any event, the system 10 preferably is designed and structured so that release of the therapeutic component occurs primarily through the exposed outer periphery of the core 22, for example as depicted by arrows 30 in FIG. 1, rather than through the top or bottom surface of the element 20.

Although the system 10 shown in FIG. 1 comprises an element 20 wherein the major surfaces of each of the first and second layer components 26, 28, has an outer periphery having a cross section in the form of a circle, it is to be appreciated that other elements having peripheries with cross-sections other than circular are also included within the scope of the present invention. For example, turning briefly to FIGS. 1a, 1b, 1c, elements 20a, 20b, 20c have top or bottom surfaces, or peripheries with cross-sections shaped as a rectangle, an oval, and an ellipse, respectively. Other shapes including other regular and irregular shapes, may be used.

Various techniques may be employed to produce the elements described and shown herein. Useful techniques include, but are not necessarily limited to, extrusion methods, co-extrusion methods, carver press method, die cutting methods, heat compression, combinations thereof and the like. Techniques for producing the cores 22 of the elements 20 include, but are not necessarily limited to, solvent-evaporation methods, phase separation methods, interfacial methods and the like.

The examples included herein are to illustrate certain aspects of the invention and are no to be considered to limit the scope of the invention.

### EXAMPLE I

Polymeric materials, Polylactic acid R 206 and R 203 from Boehringer Ingelheim, Inc. (USA), and therapeutic agents timolol maleate from Sifavitor (Italy), and mannitol (SPI Polyols) were used as received from the manufacturer.

Open sandwich elements, such as element 20 shown in FIG. 1, were prepared using a press, for example a manual Carver press with heating plates from Carver, Inc. (USA).

The term open sandwich as used herein, is a term describing an element in accordance with the invention, such as element 20, which includes laminates or layer components 26, 28 disposed on upper and/or lower surfaces of a core layer 22, and includes an exposed outer peripheral surface 34.

In this example, three-layered elements carrying timolol maleate, a highly water soluble drug, were prepared. Bottom and top layers of the element comprised pure R 206, and the core layer comprised about 50% of R 206 and about 50% of timolol maleate.

Preparation of Upper and Lower Polymeric Layers:

R 206 sheets were pre-melted in the oven at about 130° C. for about 15 minutes and compressed in a Carver press at about 2000 lb at about 64° C. for about 20 minutes. Thickness of each of the compressed sheets was approximately 0.001 inch.

Preparation of Core Layer:

A mixture of polymer and timolol maleate for the core layer was premelted and mixed at about 180° C. for about 15 minutes and compressed at about 91° C. for about 20 minutes in the Carver press with gradually applied force at about 2000 lb using a 0.005 inch Teflon separator which was used to control the layer thickness.

Open sandwiches were prepared by two different compression regimes:
1. The upper and lower polymeric layer sheets and the core layer sheet were pressed together in a Carver press for approximately 40 minutes at about 91° C. with an applied force of about 2000 lb.
2. The upper and lower polymeric layer sheets and the core layer sheet were pressed together in a Carver press for approximately 5 minutes at about 64° C. with an applied force of about 1000 lb.

Although not wishing to be bound by any particular theory of operation, it is believed that the second compression regime caused less interpenetration between the layers (polymer/drug layers interdiffusion) for example, interpenetration that is caused by high temperatures.

Next, round element samples of about 2 mm in diameter were cut from the obtained compressed sandwich sheets. Average timolol maleate content per element sample was about (15.1+/−1.9) % for the first compression regime samples and about (15.0+/−0.6)% for the second compression regime samples.

Timolol maleate release studies were performed by incubating the samples in a controlled amount of a saline solution at about 37° C. in a shaking water bath. Timolol maleate concentrations over time were analyzed by UV spectrophotometry.

Visual observation of the samples was conducted during the dissolution. Pictures were taken which appeared to show that the timolol maleate release proceeded from the periphery, through the open, exposed outer surface of the core layer. This does not fully exclude the possibility that there was diffusion of timolol maleate through the top or bottom surfaces of the core layers. However, it is believed that the amount of drug release through the first and second layer component surfaces was much less than the amount released through the exposed peripheral surface of the core.

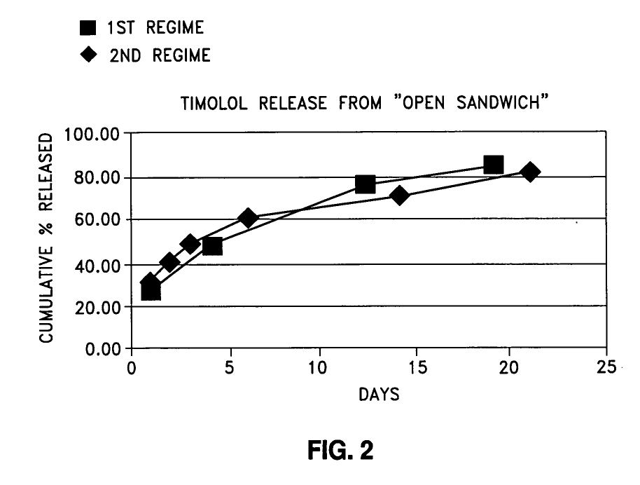

FIG. 2 shows a graph of a release profile of timolol maleate from the samples for the first and second compression regimes. As shown, the release rate profile does not show a high initial burst effect of timolol from the samples, but rather shows a more consistent, sustained, release rate. High burst effect is very typical for timolol release very fast release—continuous release—profiles of regular timolol implants.

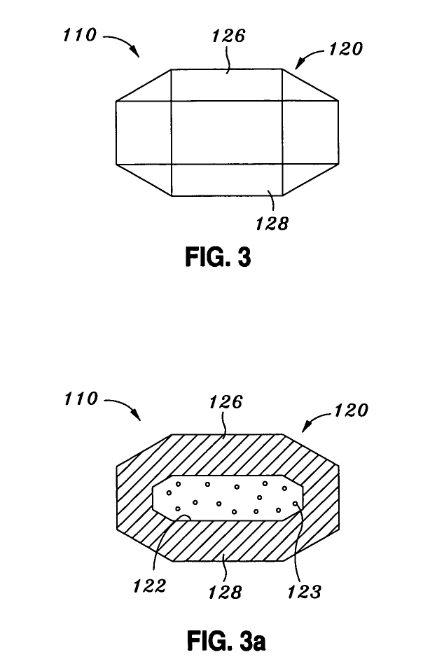

Turning now to FIGS. 3 and 3a, a side view and a cross-sectional view, respectively, of another system 110 in accordance with the present invention are shown. Unless otherwise expressly specified herein, system 110 is substantially identical to system 10, with like elements indicated by like reference numerals increased by 100.

System 110 generally comprises an element 120 comprising a first layer component 126 and a second layer component 128, which substantially surround core 122. The first and second layer components 126, 128 and core 122 are made of the same materials as described hereinabove with respect to the embodiment shown in FIG. 1. For example, first layer component 126 and second layer component 128 may comprise polymeric materials and the core 122 may comprise a therapeutically active agent mixed with a polymeric material. Unlike the embodiment shown in FIG. 1, however, in the design shown in FIGS. 3 and 3a, the core 122 is substantially entirely surrounded by, or is enclosed by, or sealed by, the first layer component 126 and the second layer component 128. In some embodiments of the invention, the first layer component 126 and the second layer component 128 effectively form a single unitary layer, for example, a single unitary layer of substantially unitary composition and/or substantially uniform thickness surrounding the core 122. It can be appreciated therefore, that whereas element 20 is sometimes elsewhere referred to herein, as having an open sandwich structure, element 120 is sometimes elsewhere referred to herein as having a sealed sandwich structure, meaning, core 122 of element 120, is substantially entirely sealed or enclosed within a different or compositionally distinguishable, polymeric layer.

For example, the core 122, which carries the therapeutic component 123, may be surrounded by pure polymeric material. This system 110 is effective in providing a delayed, and preferably in addition a sustained, drug release implant. For example, the element 120 is structured such that release of the therapeutic component 123 does not begin until a desired time period has elapsed after implantation of the element 120 into the eye. More specifically, release of the therapeutic component 123 from the core does not begin until after degradation/dissolution of the layer components 126, 128. This delayed release time period is therefore controllable by appropriate selection of the polymeric material and thickness of the layer components 126, 128.

The element 120 may be manufactured in essentially the same manner as the steps described in manufacturing the open sandwich embodiment shown in FIG. 1, with an additional step of compression, for example heat compression, of the element 120 for example, within a mold assembly.

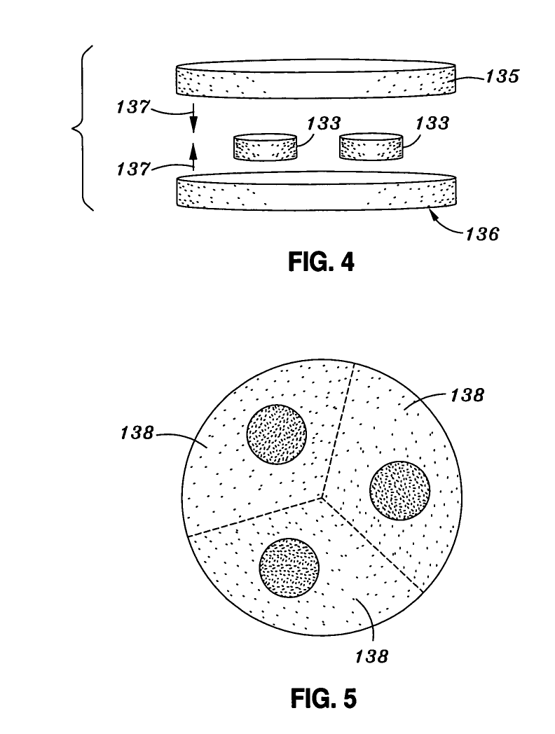

For example, preparation of sealed sandwich structures is schematically represented in FIGS. 4 and 5. FIG. 4 represents multiple timolol maleate/polymer wafers 133 which are placed in between a first layer polymer sheet 135, and a second layer polymer sheet 136. The number and arrangement of the timolol/polymer wafers 133 between the polymer sheets 135, 136 may be selected based on convenience and conservation of materials. The sheets 135 and 136 are pressed together as indicated by arrows 137, preferably while in a mold assembly, and cut, for example as indicated by dashed lines in FIG. 5, to form precursor elements 138, having a sealed sandwich structure of a desired shape and size. Note that the shape of each of the elements 138 is a combination of circular and triangular. The precursor element 138 can be implanted as is or can be further cut or otherwise molded, cut or otherwise shaped, as desired.

### Example II

More specifically, sealed sandwich elements, such as element 120 shown in FIGS. 3 and 3a were prepared as follows:

Two polymer sheets were compressed using about 0.3 g of premelted R 203 per sheet at about 48° C. (120° F.) using a 0.006 inch Teflon separator, at about 2000 lb pressure.

A mixture of about 66% timolol maleate and about 44% R 206 was premelted and mixed at about 180° C. and compressed at about 120° C. with approximately 2000 lb pressure applied using a 0.006 inch Teflon separator. Disks of about 1 cm in diameter were cut from the resulting timolol maleate polymer sheet.

Sealed sandwiches were prepared using three different compression regimes:
1. First element samples were made by compressing the three layers at approximately 82° C. and applying about 1600 lb of pressure with a 0.006 inch separator. The resulting first element samples had a thickness of about 0.007 inch.
2. Second element samples were made by compressing the three layers at about 76° C. and applying about 800 lb of pressure with a 0.005 inch separator. The resulting second element samples had a thickness of about 0.0175 inch.
3. Third element samples were made in substantially the same manner as the second element samples. However, rather than using a single upper polymer sheet and a single lower polymer sheet, the timolol maleate discs were instead compressed between two upper polymer sheets and two lower polymer sheets. This was done in order to avoid any substantial penetration of timolol maleate to outer surfaces of the element samples. Thus, using this third compression regime, third element samples were prepared having double polymer layers substantially surrounding the core layer.

A timolol maleate release study was performed by incubating the sealed sandwich samples in a controlled amount of saline solution at about 37° C. in a shaking water bath. Timolol maleate concentration was analyzed by UV spectrophotometry.

During the first day of dissolution, there was a relatively high rate of timolol maleate release in the first and second element samples. It is believed that penetration of the drug into the pure polymer layers occured during the compression step of preparing these samples. However, the third element samples, which had a double polymer layer on the outer surfaces of the core, did not show any timolol maleate release, or any substantial timolol maleate release, for over 33 days. Therefore, it is believed that penetration of timolol maleate to the surface of these samples during the compression step had been successfully avoided by providing the double layer of polymer enclosing the core. Timolol maleate release from the core had therefore been delayed by degradation of the surrounding polymeric layers and thereafter controlled by degradation of the polymeric material in the core.

In accordance with some embodiments of the invention, a soluble additive may be included in the outer polymeric layer in order to allow or to provide a pore or channel system therethrough. This embodiment may be beneficial for allowing an initial release of the therapeutic agent into the eye. The soluble additive may comprise the active agent itself, or a micron size soluble excipient, such as mannitol.

### Example III

In order to test this particular embodiment of the invention, sealed sandwich timolol maleate wafers were prepared as described in Example 11 with respect to the third element samples. However, the outer R 203 polymeric layer was provided with an effective amount of mannitol.

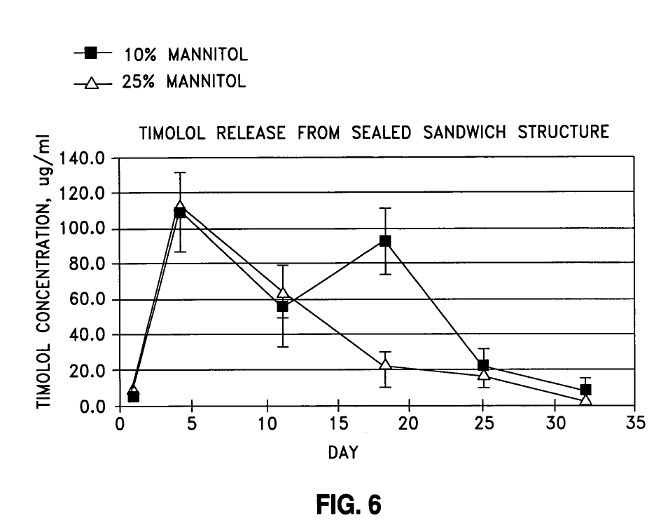

The dissolution test was carried out as described in Example II. FIG. 6 shows release rate profiles for samples having 10% mannitol added to the outer polymeric layers. As shown, the added soluble compound led to release of timolol maleate on the first day but without any significant burst effect. Therefore, providing an appropriate, effective amount of soluble additive, for example, about 10% mannitol to about 25% mannitol, in the outer polymeric layer may enable more effective control of the rate of release of the therapeutic component from the core. For example, the rate of release can be effectively controlled by either increasing the amount of additive in order to provide a faster drug release rate, or decreasing the amount of soluble additive to provide a more extended drug release rate.

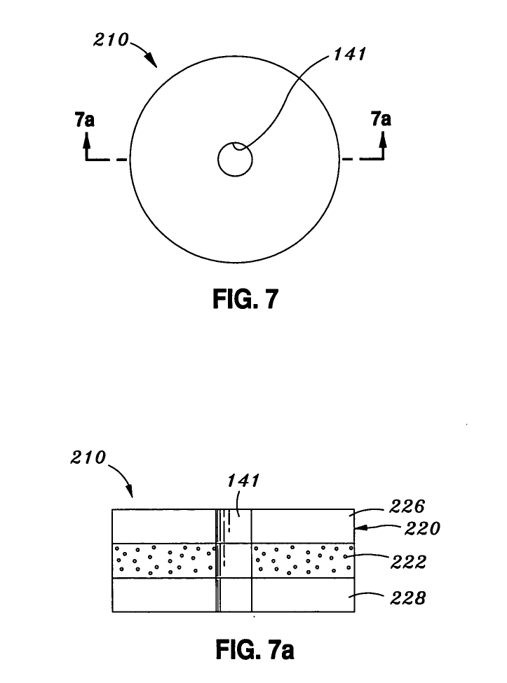

Turning now to FIGS. 7 and 7a, another system 210 in accordance with the present invention is shown. Unless otherwise expressly specified herein, system 210 is substantially identical to system 10, with like elements indicated by like reference numerals increased by 200.

System 210 generally comprises element 220. The primary difference between element 220 and element 20 is that element 220 includes a generally centrally located through aperture 141. In the particular embodiment shown in FIGS. 7 and 7a, the element 220 includes an aperture 141 provided through each of the first layer component 226, the core 222, and the second layer component 228.

Although not shown, it should be appreciated that in other embodiments of the invention, more that one aperture may be provided. In accordance with other embodiments of the invention, one or more apertures are provided through one of the first layer component 226, and/or the second layer component 228, with the core 222 is substantially solid, that is including no aperture. In some embodiments of the invention, one or more of the outer layers may be perforated and/or otherwise porous, for purposes of increasing the dissolution rate thereof, for example.

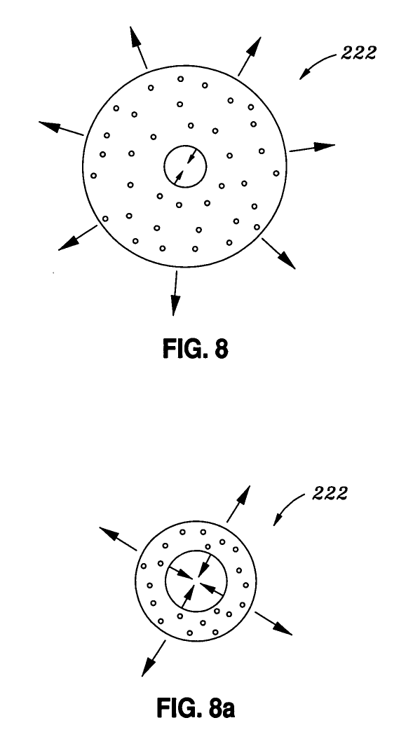

Turning now to FIGS. 8 and 8a, simplified schematic representations of the core layer 222 of element 220 are shown. FIG. 8 depicts the size and shape of the core 122 when the element 220 is initially placed in the eye, and FIG. 8a depicts the relative size and shape of the core 122 after a significant period of time has elapsed. Arrows 147 indicate the release of the therapeutic agent from the core 222, and illustrate how the rate of drug release is maintained substantially constant or consistent during dissolution/degradation of the element 220. Generally, as the outer periphery of the element 220 decreases, the inner periphery, defining aperture 141, increases, thereby substantially providing a balance, over time, of drug release from these inner and outer peripheries.

The systems of the present invention for use in the treatment of ocular conditions, such as diseases, tumors and disorders are of significant interest.

The present invention also provides methods of treating an eye, wherein the methods generally comprises the step of placing the drug delivery systems described and shown elsewhere herein, into an eye, for example, using any suitable implantation method.

For example, the method may comprise implanting the elements 20, 120, 220, at various sites in the eye. Suitable sites for implantation in the eye include the anterior chamber, posterior chamber, vitreous cavity, suprachoroidal space, subconjunctiva, episcleral, intracorneal, epicorneal and sclera. Suitable sites extrinsic to the vitreous comprise the suprachoroidal space, the pars plana and the like. The suprachoroid is a potential space lying between the inner scleral wall and the apposing choroid. Elements in accordance with the present invention that are introduced into the suprachoroid may deliver drugs to the choroid and to the anatomically apposed retina, depending upon the diffusion of the drug from the implant, the concentration of drug comprised in the implant and the like.

The elements may be introduced over or into an avascular region. The avascular region may be naturally occurring, such as the pars plana, or a region made to be avascular by surgical methods. Surgically-induced avascular regions may be produced in an eye by methods known in the art such as laser ablation, photocoagulation, cryotherapy, heat coagulation, cauterization and the like. It may be particularly desirable to produce such an avascular region over or near the desired site of treatment, particularly where the desired site of treatment is distant from the pars plana or placement of the element at the pars plana is not possible. Introduction of the over an avascular region will allow for diffusion of the drug from the element and into the inner eye and avoids diffusion of the drug into the bloodstream.

This may be more clearly understood with reference to FIG. 9, which depicts a cross-sectional view of a human eye 300 in order to illustrate the various sites that may be suitable for implantation of the elements in accordance with the present invention.

The eye 300 comprises a lens 316 and encompasses the vitreous chamber 318. Adjacent to the vitreous chamber is the optic part of the retina 322. Implantation may be into the vitreous 318, intraretinal 322 or subretinal 324. The retina 322 is surrounded by the choroid 326. Implantation may be intrachoroidal or suprachoroidal 328. Between the optic part of the retina and the lens, adjacent to the vitreous, is the pars plana 330. Surrounding the choroid 326 is the sclera 332. Implantation may be intrascleral 332 or episcleral 334. The external surface of the eye is the cornea 342. Implantation may be epicorneal 342 or intra-corneal 344. The internal surface of the eye is the conjunctiva 346. Behind the cornea is the anterior chamber 348, behind which is the lens 316. The posterior chamber 352 surrounds the lens, as shown in the figure. Opposite from the external surface is the optic nerves, and the arteries and vein of the retina. Implants into the meningeal spaces 358, the optic nerve 360 and the intraoptic nerve 361 allows for drug delivery into the central nervous system, and provide a mechanism whereby the blood-brain barrier may be crossed.

Other sites of implantation include the delivery of anti-tumor drugs to neoplastic lesions, e.g. tumor, or lesion area, e.g. surrounding tissues, or in those situations where the tumor mass has been removed, tissue adjacent to the previously removed tumor and/or into the cavity remaining after removal of the tumor. The implants may be administered in a variety of ways, including surgical means, injection, trocar, etc.

While this invention has been described with respect to various specific examples and embodiments, it is to be understood that the invention is not limited thereto and that it can be variously practiced within the scope of the following claims.

1. A drug delivery system for modified drug release into an eye comprising: an element sized and adapted for placement into an eye, said element including a core containing a therapeutic component, a first layer component located on a first side of the core, and a second layer component located on a substantially opposing second side of the core, wherein release of the therapeutic component from the core into the eye is reduced relative to a substantially identical element without the first and second layer components.

2. The system of claim 1 wherein the element has an outer peripheral surface at least a portion of which is free of the first layer component and the second layer component.

3. The system of claim 2 wherein the core has an outer peripheral surface at least a portion of which is free of the first layer component and the second layer component.

4. The system of claim 1 wherein the first layer component includes a first polymeric material and the second layer component includes a second polymeric material.

5. The system of claim 4 wherein the first polymeric material and the second polymeric material are the same material or are different materials.

6. The system of claim 1 wherein the core includes a core polymeric material.

7. The system of claim 1 wherein at least a portion of the element is biodegradable or bioerodible.

8. The system of claim 1 wherein at least one of the first layer component and the second layer component has an aperture therethrough.

9. The system of claim 1 wherein at both of the first layer component and the second layer component have apertures.

10. The system of claim 1 wherein the core has an aperture therethrough.

11. The system of claim 9 wherein the core has an aperture therethrough.

12. The system of claim 8 wherein the aperture is generally centrally located.

13. The system of claim 8 wherein the release of the therapeutic component into the eye is more constant over time relative to a substantially identical element without the aperture.

14. The system of claim 1 wherein the first and second layer components substantially surround an outer portion of the core.

15. The system of claim 1 wherein each of the element includes a gradient of concentrations of the therapeutic component that decreases outwardly from a center of the element.

16. The system of claim 1 wherein the therapeutic component comprises a drug that is soluble in the ocular environment.

17. The system of claim 16 wherein the drug comprises at least one of timolol, timolol maleate, derivatives thereof and mixtures thereof.

18. The system of claim 6 wherein the therapeutic component is substantially insoluble in the ocular environment, and the core polymeric material degrades at a faster rate in the ocular environment than the first and second layer components degrade when in the ocular environment.

19. The system of claim 1 wherein at least one of the first layer component and the second layer component contain an excipient component.

20. The system of claim 19 wherein the excipient component comprises mannitol.

21. The system of claim 17 wherein at least one of the first layer component and the second layer component comprises an excipient component.

22. The system of claim 21 wherein the excipient component comprises mannitol.

23. The system of claim 1 wherein at least one of the first layer component and the second layer component contain a therapeutic component in a concentration less than a concentration of the therapeutic component in the core.

24. A method of treating an eye comprising placing the drug delivery system of claim 1 into an eye.

25. A method of treating an eye comprising placing the drug delivery system of claim 21 into an eye.

26. A drug delivery system for modified drug release into an eye comprising. an element sized and adapted for placement into an eye, said element including a core containing a steroid component, a first layer component located on a first side of the core and including a first polymeric material, and a second layer component located on a substantially opposing second side of the core and including a second polymeric material, wherein release of the steroid component from the core into the eye is reduced relative to a substantially identical element without the first and second layer components.

27. The system of claim 25 wherein the element has an outer peripheral surface at least a portion of which is free of the first layer component and the second layer component.

28. The system of claim 26 wherein the core has an outer peripheral surface which is free of the first layer component and the second layer component.

29. The system of claim 25 wherein the first polymeric material and the second polymeric material are the same material or are different materials.

30. The system of claim 25 wherein the steroid component is dexamethasone and the first and second polymeric materials are selected from the group consisting of lactic acid polymers, glycolic acid polymers, copolymers of lactic acid and glycolic acid, and combinations thereof.

31. The system of claim 1 wherein at least a portion of the element is biodegradable or bioerodible.

32. The system of claim 25 wherein the first and second layer components substantially surround an outer portion of the core.

33. The system of claim 25 wherein at least one of the first layer component and the second layer component contain an excipient component.

34. The system of claim 25 wherein at least one of the first layer component and the second layer component contain a therapeutic component.

35. A method of treating an inflammatory condition of an eye, the method comprising placing the drug delivery system of claim 25 into an eye.

36. A method of treating an inflammatory condition of an eye, the method comprising placing the drug delivery system of claim 29 into an eye.
